(19) United States
(12) Patent Application Publication
(10) Pub. No.: US 2023/0278291 A1
(43) Pub. Date: Sep. 7, 2023

(54) RESECTIVE EPILEPSY SURGERY BRAIN SIMULATOR

(71) Applicant: THE HOSPITAL FOR SICK CHILDREN, Toronto (CA)

(72) Inventors: Grace M. Thiong'o, Toronto (CA); James M. Drake, Toronto (CA)

(21) Appl. No.: 18/116,059

(22) Filed: Mar. 1, 2023

Related U.S. Application Data
(60) Provisional application No. 63/315,262, filed on Mar. 1, 2022.

(51) Int. Cl. B29C 64/386; A61B 34/10; B33Y 50/00

(52) U.S. Cl. CPC B29C 64/386; A61B 34/10; B33Y 50/00; A61B 2034/105; G09B 23/30

(57) ABSTRACT

The present disclosure provides a resective epilepsy surgery simulator that incorporates biocompatible, dissectible materials as well as the surgical anatomy cues that would support the acquisition of the required technical skill set to ultimately broach the gap in surgical care for patients living with epilepsy. The simulator is produced by imaging a patient's brain and performing computer aided design to select the gray and white matter layers of the brain, the brain blood vessels, and the skull based on the imaging of the patient's brain and storing them in computer aided design files. These files are converted into a format readable by a 3D printer and programming the 3D printer to print the patient's brain simulator from the computer aided design files with the 3D printer containing multiple print-heads that extrude liquid polymer one multi-material layer at a time to produce a simulator of the patient's skull and brain.

### FIELD

This disclosure is directed to a functionally and anatomically specific neurosurgical simulator for the field of resective epilepsy surgery and brain dissection techniques.

### BACKGROUND

Neurosurgical simulation for competence-based surgical education utilizes technologies such as virtual reality (VR), robotics or three-dimensional (3D) printing. Surgeon trainees have preference for the hands-on training opportunities that 3D printed simulators present [1]. Dissection of brain lesions is an intricate skill: one that takes years of practice to learn. Yet it must be mastered because there is little to no room for human error in operating room [2]. Surgical simulation has been demonstrated as a method of shortening the technical skill learning curve [3]. Developing a brain dissection simulator would be an important contribution to neurosurgical education.

Material options have proliferated in the years since a patent for the first 3D printer was filed in 1984 [4], [5]. This variety of printing materials has been used surgical simulators in surgical training workshops [6], [7], [8]. Although the field of neurosurgery has developed simulators for neuroendoscopy, skull base, vascular and craniosynostosis surgeries, epilepsy surgery simulators have not been developed to the same degree [6], [7], [8], [9], [10]. Simulating resective or disconnective epilepsy surgery would require that simulators possess a complement of detailed anatomical accuracy (high physical fidelity) as well as a soft texture mimicking brain tactility (functional fidelity) [11], [12], [13]. Current simulators lack the desirable compressibility to demonstrate brain dissection techniques [16], [20], [21]. Typically for 3D printed parts, there is a desired material and a scaffolding support material.

Currently, there does not exist a hands-on surgical simulator specific for resective epilepsy surgery [20], [21]. Cadaveric brains have been used in the past; however, their use is linked to the risk of human pathogen transfer. In addition, formalin-fixation precludes cadaveric brains from being optimum brain white-matter dissection adjuncts to neurosurgical education. It would be very advantageous to provide surgical simulators specific for resective epilepsy surgery.

### SUMMARY

The present disclosure provides a cerebral hemispheric surgery simulator, also refereed to as a resective (or disconnective) epilepsy surgery simulator that incorporates dissectible materials as well as the surgical anatomy cues that would support the acquisition of the required technical skill set to ultimately broach the gap in surgical care for patients living with epilepsy.

Cerebral hemispheric surgery requires intricate neurosurgical skill and is central to preventing death and disability in selected patients with drug resistant epilepsy. Building surgical capacity and providing opportunities for patient-safe preoperative planning can likely be achieved using surgical simulation. Rapid prototyping polymers, SUP 706 and GelSupport™ manufactured by Stratasys Ltd. are traditionally the waste by-products of 3D printing but can be alternative primary material components for sustainably engineering simulator solutions for surgical education. Incorporating the use of support material as the primary material in developing a dissectible brain simulator is a promising way of advancing neurosurgical education. [21]

Thus, the present disclosure provides a process of producing a functionally and anatomically specific neurosurgical simulator, comprising:

imaging patient's brain;

performing computer aided design to select the gray and white matter layers of the brain, the brain blood vessels, and the skull based on the imaging of the patient's brain and storing them in computer aided design files;

assembling and converting the computer aided design files into a format readable by a 3D printer, programming the 3D printer to print the patient's brain simulator from said computer aided design files, said 3D printer containing multiple print-heads that extrude liquid polymer one multi-material layer at a time;

3D printing a base, the base having a size and shape commensurate with the patient's skull;

3D printing the first layer of the five lobes of the brain simultaneously with each lobe having five layers. Said five lobes comprising frontal, temporal, parietal, occipital and insular lobes, once the first layer has been printed, UV curing the first layer;

3D printing blood vessels onto the first layer such that said printed blood vessels drape the first layer, said printed blood vessels having a hollowed-out structure; and

repeating step e) for layers two, three, four and five after completion of step f) to produce an entire simulator.

The imaging of the patient's brain may be performed using Magnetic Resonance Imaging or Computed Tomography (CT) imaging.

After production of the entire simulator, the entire simulator may be encased within a layer of SUP706™ which is carefully scrapped off to reveal an underlying simulator.

The hollowed out blood vessels may be 3D printed using Stratasys® VeroMagenta RGD851 Rigid Opaque 3D printing polymer.

The first layer may be selected from a material to simulate, pia matter or intimate layer of the meninges that adheres the human brain, the second layer is selected from a polymer material to simulate grey matter, the third layer is selected from a polymer material to simulate an interface between the grey matter and white matter, the fourth layer is selected from a polymer material to simulate the white matter, and the fifth layer is selected from a polymer material to simulate the ependymal lining of the ventricles.

The first, third and fifth layers may be printed with a thickness in a range from about 0.15 mm to about 0.3 mm, and most preferably is 0.15 mm thick.

The first layer simulating pia matter, the third layer simulating the interface between the gray and white matter, and the fifth layer simulating the ependymal lining of the ventricles may be 3D printed using TissueMatrix™ polymer.

The simulator is an anatomically accurate ventricular system of the patient comprising body and temporal horns of the lateral ventricles, the third and fourth ventricles. The ventricular system are hollow cavities enclosed by layer five which simulates the ependymal lining, and wherein the hollow cavities may be prepared post 3D printing by excavating the layer of SUP706™ support material that is automatically printed to fill any gaps in a printed model. These hollow cavities are filled with water to simulate cerebrospinal fluid (CSF) in the brain.

The floor of the temporal horn of the lateral ventricle the hippocampus structure is present, and the hippocampus structure contains both gray and white matter layers.

The second layer simulating gray matter of the brain may be 3D printed using SUP706B gel-like photopolymer and being thicker over the frontal, temporal, parietal, and occipital lobes with a thickness being in the range from about 2 to about 3 mm thick and thinnest with a thickness of about 2mm over the simulated insular cortex.

The fourth layer simulating white matter may be 3D printed using SUP706B, a gel-like photopolymer, and has a thickness in a range from about 1 cm to about 3 cm.

The frontal, temporal, parietal, occipital and insular lobes replicate the gyri and sulci of a human brain, and wherein the temporal and parietal opercula are present in the simulator.

The imaging may be of a patient with intractable epilepsy of neonatal stroke etiology such that when the simulator is 3D printed from the imaging, the frontal and parietal operculum are absent in keeping with the gliotic degeneration pathophysiology present in patients with intractable epilepsy, and wherein once the 3D printing phase is complete, in order to simulate a gliotic membrane covering a defect on the stroke-infarcted brain region, a 0.3 mm layer of silicone material, Ecoflex™ 00-10, is prepared having dimensions corresponding to a gap formed in the 3D printed corresponding to the defect in the stroke-infarcted brain region, and then affixed thereto followed by air drying.

The second layer simulating gray matter of the brain may be 3D printed using GelSupport™ rapid prototyping photopolymer (Shore A material property score, 30) which is a mixture of Sup706, GelMatrix™ and Agilus30, said second layer being thicker over the frontal, temporal, parietal, and occipital lobes with a thickness being in the range from about 2 to about 3 mm thick and thinnest with a thickness of about 2mm over the simulated insular cortex.

The fourth layer simulating white matter may be 3D printed using GelSupport™ which is a gel-like rapid prototyping photopolymer (Shore A material property score, 30), and which is a mixture of Sup706, GelMatrix™ and Agilus30, and has a thickness in a range from about 1 cm to about 3 cm.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 illustrates a first embodiment of a brain phantom of the left cerebral hemisphere with the lobes of the brain, blood vessels and ventricles labelled.

FIG. 2 is an axial cross-sectional comparison of the internal anatomy of the first embodiment of FIG. 1 (SUP706™) and a second embodiment (GelSupport™) against that of a cadaveric brain cross-section.

FIG. 3 is a lateral view of the simulator with the main trunk of the middle cerebral artery blood vessel perched upon the insular lobe.

FIG. 4 shows the second embodiment of the present cerebral hemispheric surgery (resective/disconnective epilepsy surgery) simulator next to a neuronavigation reference frame.

FIG. 5 shows the simulators ability to register onto neuro-navigation and point to a desired target. In this case the surgeon is navigating to the tip of the temporal horn of the lateral ventricle which is marked by crosshairs.

FIG. 6 shows the appearance of the second embodiment with internal anatomy revealed. The labelled blood vessels served as visual surgical cues during cerebral hemispheric epilepsy surgery simulation.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described so as to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps, or components.

As used herein, the term “exemplary” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

### EMBODIMENT NUMBER 1

### Anatomical layers

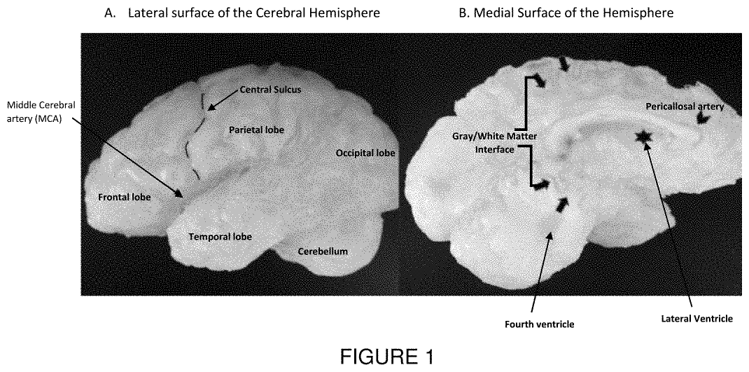

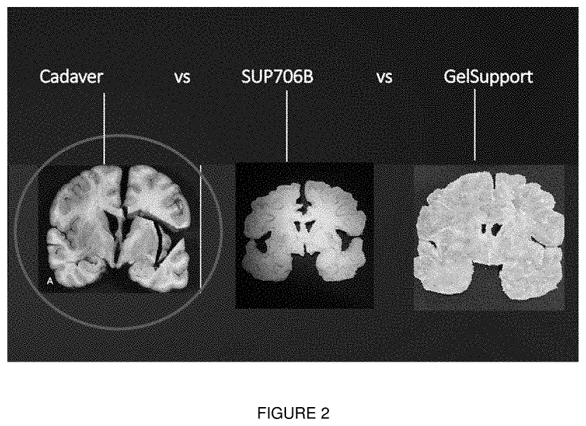

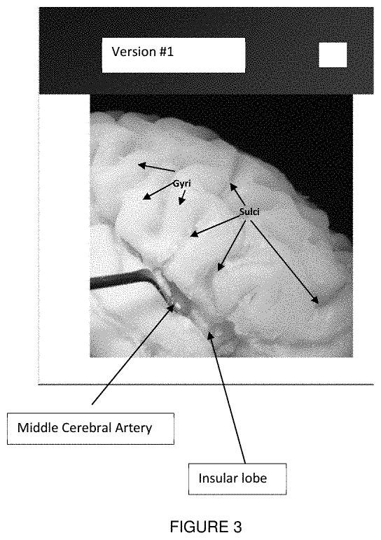

The cerebral hemispheric simulator (resective epilepsy surgery simulator) described comprises five lobes of the brain each with five layers. FIG. 1 illustrates this first embodiment of a brain phantom of the left cerebral hemisphere with the lobes of the brain, blood vessels and ventricles labelled. Additionally, pertinent neurovasculature is contained and described herein. The anatomically accurate lobes include the frontal, temporal, parietal, occipital and insular lobes. Each of the lobes has the five distinct layers described below. All five layers are preferably printed using a J750 Digital Anatomy 3D printer, which is currently, the only 3D printer that can utilize TissueMatrix™. In addition, the J750 Digital Anatomy printer was selected for the five layers owing to its ability to print multi-material constructs simultaneously and with a high level of detail (the minimum print thickness possible is 14 microns). The layers are described from the most superficial to the deepest layer. FIG. 2 shows an axial cross-sectional comparison of the internal anatomy of the first embodiment of FIG. 1 (SUP706B™). FIG. 3 is a lateral view of this first embodiment of the simulator with the main trunk of the middle cerebral artery blood vessel perched upon the insular lobe.

It can have a thickness in a range from about 0.15 mm to about 0.3 mm, and most preferably is 0.15 mm thick.

The outermost layer, or layer 1 simulates pia matter, is about 0.15 mm thick and is 3D printed using TissueMatrix™, a commercially available polymer compatible with the J750 Digital Anatomy 3D printer. The material properties relied upon by the simulator are the lowest formulation (0.262 +/−0.0004 N/m²). TissueMatrix™ is marketed as the softest commercially available material, at present. This first anatomical layer mimics the intimate layer of the meninges that adheres the human brain. The layers are preferably deposited simultaneously which can be done advantageously using the J750 printer. The layers of the simulator can be printed from the bottom up, thereby allowing deposition of all the materials at the same time. The horizontal build platform layers are deposited at a thickness of about 14 microns at a time until the entire construct is complete.

Layer two of the invention anatomically mimics the gray matter of the brain and is made of SUP706B gel-like photopolymer (Shore O material property score=15). It is commercially available and printable on a J750 Digital Anatomy 3D printer. The simulated gray matter has a thickness in the range from about 2 to 20 about 3 mm thick, being thicker over the frontal, temporal, parietal, and occipital lobes and thinnest (about 2 mm) over the insular cortex. Table 1 shows the composition of SUP706.

### SUP706 COMPOSITION:

| CAS | Component name | Percent |
|---|---|---|
| 57-55-6 | 1,2-Propylene glycol | <35 |
| 25322-68-3 | Polyethylene glycol | <30 |
| — | Acrylic monomer | <25 |
| 947-19-3 | Methanone, (1- | <2 |
| | hydroxycylcohexyl) | |
| | phenyl- | |

Table 1

Layer three marks the interface between the gray and white matter. It is similar in dimension and architecture to the first layer, the pia matter. It is preferably 3D printed using TissueMatrix™ and has a thickness in a range from about 0.15 mm to about 0.3 mm, and most preferably is 0.15 mm thick.

Layer four, the white matter, comprises the bulk of simulator and is made of SUP706B, a gel-like photopolymer that is commercially available and printable on a J750 Digital Anatomy 3D printer. The corpus callosum and internal capsule components of the white matter are visible on cross-sectional anatomy of the simulator. The white matter layer fills the entire volume between the overlying gray matter (layer three) and the underlying ependymal lining (layer five), with a thickness ranging from about 1 to about 3 centimeters. The innermost layer, layer five, is the ependymal lining of the ventricles. Like layers one and three it can have a thickness in the range from about 0.15 mm to about 0.3 mm, and most preferably is about 0.15 mm thick, and commercially available as polymer, TissueMatrix™.

The cerebral ventricles

Embedded within the simulator is an anatomically accurate ventricular system comprising the body and temporal horns of the lateral ventricles, the third and fourth ventricles. The ventricular system is a cavity enclosed by layer five (the ependymal lining). These are hollow cavities prepared in the simulator post-printing processing phase and filled with water to simulate cerebrospinal fluid (CSF) in the brain. The hollow cavities are prepared by excavating the reduntant SUP706™ support material that is automatically printed to fill any gaps present in a printed model.

At the floor of the temporal horn of the lateral ventricle the hippocampus structure is present. The hippocampus contains both gray and white matter layers similar to the description above.

Brain Blood vessels

Neurovasculature specific to epilepsy surgery, specifically, a pair of the pericallosal arteries overlying the corpus callosum, the middle cerebral artery and its insular branches and the circle of Willis are incorporated in the simulator. The blood vessels drape the brain simulator above layer one. These blood vessels are hollowed out structures that are 3D printed using commercially available Stratasys® VeroMagenta RGD851 Rigid Opaque 3D printing polymer.

Lobes of the brain

All five lobes: the frontal, temporal, parietal, occipital and insular lobes replicate the gyri and sulci of a human brain. The temporal and parietal opercula are present in embodiment 1.

A base that the cerebral hemispheric surgery simulator rests on is created using commercially available, biocompatible, sterilizable, ABS M30i thermoplastic. A fused deposition modelling (FDM) technique 3D printer was used for this step as this was very convenient for this purpose, however it will be appreciated that any 3D printer can be used to fabricate the base.

Having described above the various layers and commercially available materials used to 3D print them and having described for the example the cerebral ventricles, brain blood vessels and lobes of the brain, the process for 3D printing the brain simulator will now be described.

First, once a patient is identified requiring resective epilepsy surgery, the surgeons prefer to have a patient anatomically specific neurosurgical simulator which emulates the patient's brain in order to practise the surgery prior to operating on the patient. This requires imaging of the brain, or at least that part of the brain in which the surgery will take place. Embodiment one was designed using brain Magnetic Resonance Imaging (MRI) of an adult male patient obtained from 3D slicer (https://www.slicer.org/). 3D slicer™ is a free, open source and multi-platform software package for medical, biomedical, and related imaging research. Slicer is a trademark of Brigham and Women's Hospital (BWH) and is intended for research use. Details of licence can be found on the link: https://slicer.readthedocs.io/en/latest/userguide/about.html//license

Subsequently, computer aided design (CAD) was performed using Materialise™ software. CAD selected the gray and white matter layers of the brain, the brain blood vessels, and the skull by a segmentation process. The CAD files were assembled and converted to Standard Tessellation Language (STL), which is a 3D printer-friendly format, that directly be read and printed by any 3D printer model. The CAD segmented assembly is described in detail above.

Once the 3D printer has been programmed to print the patient's brain simulator, the following steps are followed to 3D print the simulator. First, a base on which the five (5) different layers is produced. Once the base has been produced, the patient's brain simulator is then 3D printed. The layers are printed simultaneously owing to the multi-material properties of the J750 Digital Anatomy Printer. The printer contains multiple print-heads that extrude liquid polymer one multi-material layer at a time, with each layer subsequently being cured by UV light before another is extruded onto the build platform. This process is repeated until the simulator is complete. The entire simulator is encased within a layer of SUP706™ which is carefully scrapped off to reveal the underlying simulator. An advantage of the J750 printer is that it can deposit all the layers simultaneously due to the multiplicity of print heads.

### EMBODIMENT NUMBER 2

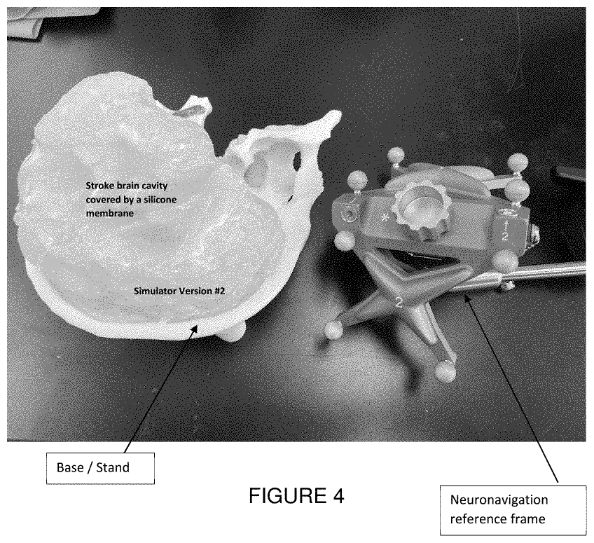

Embodiment two was fashioned after a fourteen-year-old with intractable epilepsy of neonatal stroke etiology. FIG. 4 illustrated the complete embodiment number two, perched on a base, alongside a neuronavigation reference frame. The images were obtained following research ethics board approval. Subsequently, computer aided design (CAD) was performed using Materialise™ software. CAD selected the gray and white matter layers of the brain, the brain blood vessels, and the skull by a segmentation process. The CAD files were assembled and converted to Standard Tessellation Language (STL), which is a 3D printer-friendly format, that can directly be read and printed by any 3D printer model. The CAD segmented assembly is described in detail below.

Anatomical layers

The cerebral hemispheric simulator (resective epilepsy surgery simulator) described comprises five lobes of the brain each with five layers Additionally, pertinent neurovasculature is contained and described herein. The anatomically accurate lobes include the frontal, temporal, parietal, occipital and insular lobes. Each of the lobes has the five distinct layers described below. All five layers are printed using a J750 Digital Anatomy 3D printer, which is currently, the only 3D printer that can utilize TissueMatrix™. In addition, the J750 Digital Anatomy printer was selected for the five layers owing to its ability to print multi-material constructs simultaneously and with a high level of detail (the minimum print thickness possible is 14 microns). The layers are described from the most superficial to the deepest layer.

The first or outermost layer, pia matter, is a 0.15 mm thick layer of TissueMatrix™, a commercially available polymer compatible with a J750 Digital Anatomy 3D printer. The material properties relied upon by the simulator are the lowest formulation (0.262+/−0.0004 N/m²). This first anatomical layer mimics the intimate layer of the meninges that adheres the human brain. Layer one can have a thickness in the range from about 0.15 mm to about 0.3 mm, and most preferably is about 0.15 mm thick, and is 3D printed using commercially available polymer, TissueMatrix™.

Layer two of the invention anatomically mimics the gray matter of the brain and is 3D printed using GelSupport™ a gel-like rapid prototyping photopolymer (Shore A material property score, 30). GelSupport™ is a mixture of Sup706, GelMatrix™ and Agilus30 in undisclosed ratios. It is commercially available and printable on a J750 Digital Anatomy 3D printer. The simulated gray matter has a thickness in the range from about 2 to about 3 mm thick, being thicker over the frontal, temporal, parietal, and occipital lobes and thinnest (about 2 mm) over the insular cortex.

Layer three marks the interface between the gray and white matter. It is similar in dimension and architecture to the first layer, the pia matter. It is preferably 3D printed using TissueMatrix™and has a thickness in a range from about 0.15 mm to about 0.3 mm, and most preferably is 0.15 mm thick.

Layer four, the white matter, comprises the bulk of simulator and is 3D printed using GelSupport™, a gel-like photopolymer that is commercially available and printable on a J750 Digital Anatomy 3D printer. The corpus callosum and internal capsule components of the white matter are visible on cross-sectional anatomy of the simulator. The white matter layer fills the entire volume between the overlying gray matter (layer three) and the underlying ependymal lining (layer five), with a thickness ranging from about 1 to about 3 centimeters.

The innermost layer, layer five, is the ependymal lining of the ventricles. Like layers one and three it can have a thickness in the range from about 0.15 mm to about 0.3 mm, and most preferably is about 0.15 mm thick, and is 3D printed using commercially available polymer, TissueMatrix™.

The cerebral ventricles

Embedded within the simulator is an anatomically accurate ventricular system comprising the body and temporal horns of the lateral ventricles, the third and fourth ventricles. The ventricular system is a cavity enclosed by layer five (the ependymal lining).

These are hollow cavities prepared in the simulator post-printing processing phase and filled with water to simulate cerebrospinal fluid (CSF) in the brain. At the floor of the temporal horn of the lateral ventricle the hippocampus structure is present. The hippocampus contains both gray and white matter layers similar to the description above.

Brain Blood vessels

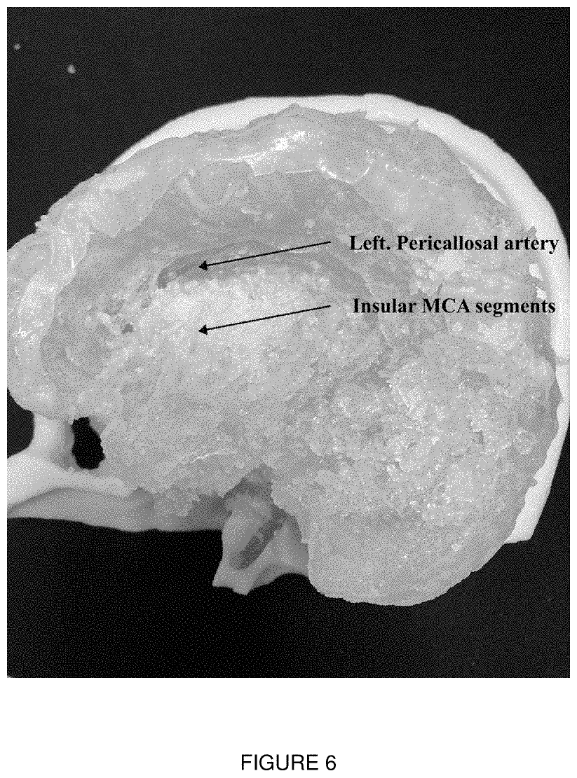

Neurovasculature specific to epilepsy surgery, specifically, a pair of the pericallosal arteries overlying the corpus callosum, the middle cerebral artery and its insular branches and the circle of Willis are incorporated in the simulator. The blood vessels drape the brain simulator above layer one. These blood vessels are hollowed out structures that are 3D printed using commercially available Stratasysφ VeroMagenta RGD851 Rigid Opaque 3D printing polymer. FIG. 6 shows the blood vessels revealed in embodiment number two, after surgical simulation has been performed.

Lobes of the brain

All five lobes: the frontal, temporal, parietal, occipital and insular lobes replicate the gyri and sulci of a human brain. The temporal and parietal opercula are absent in embodiment no. 2. The frontal and parietal operculum in stroke-brain embodiments (embodiment #2) are absent in keeping with the gliotic degeneration pathophysiology. Each of the lobes has the five distinct layers described above. A base for the cerebral hemispheric surgery simulator rests on is created using commercially available, biocompatible, sterilizable, ABS M30i thermoplastic. A fused deposition modelling (FDM) technique 3D printer was used for this.

A layer of silicone material of about 0.3 mm of Ecoflex™ 00-10, forms the gliotic membrane remnant of the infarcted region of the brain. This layer is hand-made by spreading liquid Ecoflex™ 00-10 on a flat pan and drying it prior to cutting out a shape similar to the gap formed by the 3D printed cavity. It is then fixed to the simulator using liquid Ecoflex™ 00-10 on the edges and allowed to air dry. GelSupport™ embodiments that do not simulate a stroked brain may omit this step.

Once the base has been produced, the patient's brain simulator is then 3D printed. The base having a size and shape commensurate with the patient's skull as determined from the imaging.

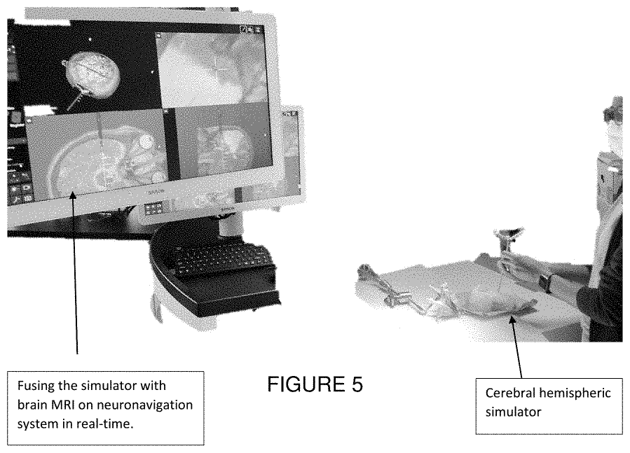

The layers are printed simultaneously owing to the multi-material property of the J750 Digital Anatomy Printer. The printer contains multiple print-heads that extrude liquid polymer one multi-material layer at a time, with each layer subsequently being cured by UV light before another is extruded onto the build platform. This process is repeated until the simulator is complete. The entire simulator is encased within a layer of SUP706™ which is carefully scrapped off to reveal the underlying simulator. In this regard, the printer automatically prints support material on all edges that have a 45-degree overhang. This is done for stability and since the brain simulator is largely a curvy structure, it ends up being encased in the external support material. The simulator once assembled is compatible with neuronavigation software as is illustrated in FIG. 5, which shows merging of the simulator with the patient's brain MRI scans.

### Comparison of Embodiments Numbers 1 and 2

The purpose of embodiment number one is to harness the break-away properties of SUP706™, while utilizing the sturdier nature of TissueMatrix™ and Agilus both to capture the functional fidelity necessary to simulate disconnective epilepsy surgical procedures, when the brain anatomy is in its normal, complete state.

The purpose of embodiment number two is to improve on the robustness of embodiment 1 by simulating actual epilepsy patient pathology as well as incorporating an even softer material, GelSupport™, in order to approximate the feel of the simulator to that of an actual patient's brain. Brain suction and dissection is thus easier for a surgeon to simulate with embodiment two.

Therefore, embodiment one is used when the brain anatomy is in its normal, complete state, while the simulator of embodiment no. 2 is produced for a surgeon to practise resective epilepsy surgery on a patient with intractable epilepsy prior to surgery on the patient themselves.

In an embodiment there is provided a process of producing a functionally and anatomically specific neurosurgical simulator, comprising:

a) imaging patient's brain;

b) performing computer aided design to select the gray and white matter layers of the brain, the brain blood vessels, and the skull and storing them in files;

c) assembling and converting the computer aided design files into a format readable by a 3D printer, programming the 3D printer to print the patient's brain simulator from said computer aided design files, said 3D printer containing multiple print-heads that extrude liquid polymer one multi-material layer at a time;

d) 3D printing a base, the base having a size and shape commensurate with the patient's skull;

e) 3D printing the first layer of the five lobes of the brain simultaneously with each lobe having five layers. Said five lobes comprising frontal, temporal, parietal, occipital and insular lobes, once the first layer has been printed, UV curing the first layer;

f) 3D printing blood vessels onto the first layer such that said printed blood vessels drape the first layer, said printed blood vessels having a hollowed-out structure; and

g) repeating step e) for layers two, three, four and five after completion of step f) to produce an entire simulator.

In an embodiment the imaging of the patient's brain is performed using Magnetic Resonance Imaging or Computed Tomography (CT) imaging.

In an embodiment, after production of the entire simulator, encasing the entire simulator within a layer of SUP706™ which is carefully scrapped off to reveal an underlying simulator.

In an embodiment, the hollowed out blood vessels are 3D printed using Stratasys® VeroMagenta RGD851 Rigid Opaque 3D printing polymer.

In an embodiment the first layer is selected from a material to simulate, pia matter or intimate layer of the meninges that adheres the human brain, said second layer is selected from a polymer material to simulate grey matter, said third layer is selected from a polymer material to simulate an interface between the grey matter and white matter, said fourth layer is selected from a polymer material to simulate the white matter, and wherein said fifth layer is selected from a polymer material to simulate the ependymal lining of the ventricles.

In an embodiment, the first, third and fifth layers are printed with a thickness in a range from about 0.15 mm to about 0.3 mm.

In an embodiment, the first, third and fifth layers are 3D printed with a thickness of about 0.15 mm.

In an embodiment, the first layer simulating pia matter, the third layer simulating the interface between the gray and white matter, and the fifth layer simulating the ependymal lining of the ventricles are 3D printed using TissueMatrix™ polymer.

In an embodiment, the simulator is an anatomically accurate ventricular system of the patient comprising body and temporal horns of the lateral ventricles, the third and fourth ventricles.

In an embodiment, the ventricular system are hollow cavities enclosed by layer five which simulates the ependymal lining, and wherein the hollow cavities are prepared post 3D printing by excavating the layer of SUP706™ support material that is automatically printed to fill any gaps in a printed model.

In an embodiment, the method includes filling the hollow cavities with water to simulate cerebrospinal fluid (CSF) in the brain.

In an embodiment, the second layer simulating gray matter of the brain is 3D printed using SUP706B gel-like photopolymer and being thicker over the frontal, temporal, parietal, and occipital lobes with a thickness being in the range from about 2 to about 3 mm thick and thinnest with a thickness of about 2 mm over the simulated insular cortex.

In an embodiment, the fourth layer simulating white matter is 3D printed using SUP706B, a gel-like photopolymer, and has a thickness in a range from about 1 cm to about 3 cm.

In an embodiment, the frontal, temporal, parietal, occipital and insular lobes replicate the gyri and sulci of a human brain, and wherein the temporal and parietal opercula are present in the simulator.

In an embodiment, the imaging is of a patient with intractable epilepsy of neonatal stroke etiology such that when the simulator is 3D printed from the imaging, the frontal and parietal operculum are absent in keeping with the gliotic degeneration pathophysiology present in patients with intractable epilepsy, and wherein once the 3D printing phase is complete, in order to simulate a gliotic membrane covering a defect on the stroke-infarcted brain region, a 0.3 mm layer of silicone material, Ecoflex™ 00-10, is prepared having dimensions corresponding to a gap formed in the 3D printed corresponding to the defect in the stroke-infarcted brain region, and then affixed thereto followed by air drying

In an embodiment, the second layer simulating gray matter of the brain is 3D printed using GelSupport™ rapid prototyping photopolymer (Shore A material property score, 30) which is a mixture of Sup706, GelMatrix™ and Agilus30, said second layer being thicker over the frontal, temporal, parietal, and occipital lobes with a thickness being in the range from about 2 to about 3 mm thick and thinnest with a thickness of about 2 mm over the simulated insular cortex.

In an embodiment, the fourth layer simulating white matter is 3D printed using GelSupport™ which is a gel-like rapid prototyping photopolymer (Shore A material property score, 30), and which is a mixture of Sup706, GelMatrix™ and Agilus30, and has a thickness in a range from about 1 cm to about 3 cm.

While the present process of producing the anatomical simulator of the patient's brain and skull has been illustrated using Magnetic Resonance Imaging it will be appreciated that other types of imaging may be used, including but not limited to Computed Tomography (CT) imaging.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

### REFERENCES

[1] Hochman, J. B., Rhodes, C., Kraut, J., Pisa, J. & Unger, B. End User Comparison of Anatomically Matched 3-Dimensional Printed and Virtual Haptic Temporal Bone Simulation: A Pilot Study. Otolaryngol.-Head Neck Surg. (United States) (2015) doi:10.1177/0194599815586756.

[2] Malone, H. R. et al. Simulation in neurosurgery: A review of computer-based simulation environments and their surgical applications. Neurosurgery (2010) doi:10.1227/NEU.0b013e3181ee46d0.

[3] Milburn, J. A., Khera, G., Hornby, S. T., Malone, P. S. & Fitzgerald, J. E. Introduction, availability and role of simulation in surgical education and training: review of current evidence and recommendations from the Association of Surgeons in Training. International journal of surgery (London, England) (2012) oi:10.1016/j.ijsu.2012.05.005.

[4] Hull, C. W. Apparatus for Production of Three-Dimensional Objects By Stereolithography. Patent (1984).

[5] Whitaker, M. The history of 3D printing in healthcare. Bull. R. Coll. Surg. Engl. (2014) doi:10.1308/147363514x13990346756481.

[6] Breimer, G. E., Bodani, V., Looi, T. & Drake, J. M. Design and evaluation of a new synthetic brain simulator for endoscopic third ventriculostomy. J. Neurosurg. Pediatr. (2015) doi:10.3171/2014.9.PEDS1447.

[7] Bodani, V. P., Breimer, G. E., Haji, F. A., Looi, T. & Drake, J. M. Development and evaluation of a patient-specific surgical simulator for endoscopic colloid cyst resection. J. Neurosurg. (2019) doi:10.3171/2019.4.jns183184.

[8] Eastwood, K. W. et al. Development of synthetic simulators for endoscope-assisted repair of metopic and sagittal craniosynostosis. J. Neurosurg. Pediatr. (2018) doi:10.3171/2018.2.peds18121.

[9] Hsieh, T. Y., Cervenka, B., Dedhia, R., Strong, E. B. & Steele, T. Assessment of a patient-specific, 3-dimensionally printed endoscopic sinus and skull base surgical model. JAMA Otolaryngol.-Head Neck Surg. (2018) doi:10.1001/jamaoto.2018.0473.

[10] Lin, J. et al. Using Three-Dimensional Printing to Create Individualized Cranial Nerve Models for Skull Base Tumor Surgery. World Neurosurg. (2018) doi:10.1016/j.wneu.2018.07.236.

[11] Maran, N. J. & Glavin, R. J. Low- to high-fidelity simulation—A continuum of medical education? Medical Education, Supplement (2003) doi:10.1046/j.1365-2923.37.s1.9.x.

[12] Munshi, F., Lababidi, H. & Alyousef, S. Low- versus high-fidelity simulations in teaching and assessing clinical skills. Journal of Taibah University Medical Sciences (2015) doi:10.1016/j.jtumed.2015.01.008.

[13] Miller, R. B. Psychological Considerations In The Design Of Training Equipment. Distribution (1954).

[14] Bova, F. J. et al. Mixed-reality simulation for neurosurgical procedures. Neurosurgery (2013) doi:10.1227/NEU.0000000000000113.

[15] Ryan, J. R., Chen, T., Nakaji, P., Frakes, D. H. & Gonzalez, L. F. Ventriculostomy Simulation Using Patient-Specific Ventricular Anatomy, 3D Printing, and Hydrogel Casting. World Neurosurg. (2015) doi:10.1016/j.wneu.2015.06.016.

[16] Sase, K., Fukuhara, A., Tsujita, T. & Konno, A. GPU-accelerated surgery simulation for opening a brain fissure. ROBOMECH J. (2015) doi:10.1186/s40648-015-0040-0.

[17] Bernardo, A. Virtual Reality and Simulation in Neurosurgical Training. World Neurosurg. (2017) doi:10.1016/j.wneu.2017.06.140.

[18] Porte, M. C., Xeroulis, G., Reznick, R. K. & Dubrowski, A. Verbal feedback from an expert is more effective than self-accessed feedback about motion efficiency in learning new surgical skills. Am. J. Surg. (2007) doi:10.1016/j.amjsurg.2006.03.016.

[19] Wongsriruksa, S., Howes, P., Conreen, M. & Miodownik, M. The use of physical property data to predict the touch perception of materials. Mater. Des. (2012) doi:10.1016/j.matdes.2012.05.054.

[20] Thiong'o, G. M., Bernstein, M., & Drake, J. M. (2021). 3D printing in neurosurgery education: a review. 3D Printing in Medicine, 7(1), 1-6.

[21] Thiong'o, G. M., Looi, T., & Drake, J. M. (2021, November). Application of 3D Printing Support Material for Neurosurgical Simulation. In 2021 43^{rd} Annual International Conference of the IEEE Engineering in Medicine & Biology Society (EMBC)(pp. 4169-4173). IEEE.

Therefore, what is claimed is:

1. A process of producing a functionally and anatomically specific neurosurgical simulator, comprising:
a) imaging patient's brain;
b) performing computer aided design to select the gray and white matter layers of the brain, the brain blood vessels, and the skull based on the imaging of the patient's brain and storing them in computer aided design files;
c) assembling and converting the computer aided design files into a format readable by a 3D printer, programming the 3D printer to print the patient's brain simulator from said computer aided design files, said 3D printer containing multiple print-heads that extrude liquid polymer one multi-material layer at a time;
d) 3D printing a base, the base having a size and shape commensurate with the patient's skull;
e) 3D printing the first layer of the five lobes of the brain simultaneously with each lobe having five layers. Said five lobes comprising frontal, temporal, parietal, occipital and insular lobes, once the first layer has been printed, UV curing the first layer;
f) 3D printing blood vessels onto the first layer such that said printed blood vessels drape the first layer, said printed blood vessels having a hollowed-out structure; and
g) repeating step e) for layers two, three, four and five after completion of step f) to produce an entire simulator.

2. The method according to claim 1, wherein the imaging of the patient's brain is performed using Magnetic Resonance Imaging or Computed Tomography (CT) imaging.

3. The method according to claim 1, wherein after production of the entire simulator, encasing the entire simulator within a layer of SUP706™ which is carefully scrapped off to reveal an underlying simulator.

4. The method according to claim 1, wherein said hollowed out blood vessels are 3D printed using Stratasys® VeroMagenta RGD851 Rigid Opaque 3D printing polymer.

5. The method according to claim 1, wherein said first layer is selected from a material to simulate, pia matter or intimate layer of the meninges that adheres the human brain, said second layer is selected from a polymer material to simulate grey matter, said third layer is selected from a polymer material to simulate an interface between the grey matter and white matter, said fourth layer is selected from a polymer material to simulate the white matter, and wherein said fifth layer is selected from a polymer material to simulate the ependymal lining of the ventricles.

6. The method according to claim 1, wherein said first, third and fifth layers are printed with a thickness in a range from about 0.15 mm to about 0.3 mm.

7. The method according to claim 1, wherein said first, third and fifth layers are 3D printed with a thickness of about 0.15 mm.

8. The method according to claim 1, wherein said first layer simulating pia matter, the third layer simulating the interface between the gray and white matter, and the fifth layer simulating the ependymal lining of the ventricles are 3D printed using TissueMatrix™ polymer.

9. The method according to claim 1, wherein said simulator is an anatomically accurate ventricular system of the patient comprising body and temporal horns of the lateral ventricles, the third and fourth ventricles.

10. The method according to claim 9, wherein the ventricular system are hollow cavities enclosed by layer five which simulates the ependymal lining, and wherein the hollow cavities are prepared post 3D printing by excavating the layer of SUP706™ support material that is automatically printed to fill any gaps in a printed model.

11. The method according to claim 10, including filling the hollow cavities with a fluid selected to simulate cerebrospinal fluid (CSF) in the brain.

12. The method according to claim 10, including filling the hollow cavities with aqueous solution to simulate cerebrospinal fluid (CSF) in the brain.

13. The method according to claim 12, wherein the aqueous solution is water.

14. The method according to claim 9, wherein a floor of the temporal horn of the lateral ventricle the hippocampus structure is present, and wherein the hippocampus structure contains both gray and white matter layers.

15. The method according to claim 1, wherein said second layer simulating gray matter of the brain is 3D printed using SUP706B gel-like photopolymer and being thicker over the frontal, temporal, parietal, and occipital lobes with a thickness being in the range from about 2 to about 3 mm thick and thinnest with a thickness of about 2mm over the simulated insular cortex.

16. The method according to claim 1, wherein said fourth layer simulating white matter is 3D printed using SUP706B, a gel-like photopolymer, and has a thickness in a range from about 1 cm to about 3 cm.

17. The method according to claim 1, wherein the frontal, temporal, parietal, occipital and insular lobes replicate the gyri and sulci of a human brain, and wherein the temporal and parietal opercula are present in the simulator.

18. The method according to claim 1, wherein said imaging is of a patient with intractable epilepsy of neonatal stroke etiology such that when the simulator is 3D printed from the imaging, the frontal and parietal operculum are absent in keeping with the gliotic degeneration pathophysiology present in patients with intractable epilepsy, and wherein once the 3D printing phase is complete, in order to simulate a gliotic membrane covering a defect on the stroke-infarcted brain region, a 0.3 mm layer of silicone material, Ecoflex™ 00-10, is prepared having dimensions corresponding to a gap formed in the 3D printed corresponding to the defect in the stroke-infarcted brain region, and then affixed thereto followed by air drying.

19. The method according to claim 18, wherein said second layer simulating gray matter of the brain is 3D printed using GelSupport™ rapid prototyping photopolymer (Shore A material property score, 30) which is a mixture of Sup706, GelMatrix™ and Agilus30, said second layer being thicker over the frontal, temporal, parietal, and occipital lobes with a thickness being in the range from about 2 to about 3 mm thick and thinnest with a thickness of about 2 mm over the simulated insular cortex.

20. The method according to claim 18, wherein said fourth layer simulating white matter is 3D printed using GelSupport™ which is a gel-like rapid prototyping photopolymer (Shore A material property score, 30), and which is a mixture of Sup706, GelMatrix™ and Agilus30, and has a thickness in a range from about 1 cm to about 3 cm.

21. A functionally and anatomically specific neurosurgical simulator produced by the method of claim 1.
